(19) United States
(12) Patent Application Publication
(10) Pub. No.: US 2005/0019284 A1
(43) Pub. Date: Jan. 27, 2005

(54) Stabilized retinoid make-up compositions

(76) Inventors: Victoria Van Leeuwen, Le Vaudreuil (FR); Marie-Paule Robert, Crema (IT); Patricia Manissier, Levallois-Perret (FR)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/486,096

(22) PCT Filed: Aug. 14, 2002
(86) PCT No.: PCT/IB02/03625

(30) Foreign Application Priority Data
Aug. 14, 2001 (FR) 01 10809

(51) Int. Cl. A61K 7/021

(52) U.S. Cl. 424/63

(57) ABSTRACT

The present invention relates to cosmetic compositions having good physical and chemical stability, comprising a retinoid and at least one coloured pigment selected from metal oxides, the said pigments being treated (or coated) with one or more hydrophilic or lipophilic components.

### BACKGROUND OF THE INVENTION

The present invention relates to cosmetic compositions having good physical and chemical stability, comprising a retinoid and at least one coloured pigment selected from metal oxides, the said pigments being treated (or coated) with one or more hydrophilic or lipophilic components.

Over the past few years, cosmetic products containing retinoids have been of increasing interest and have become highly successful with consumers. Retinoids such as retinoic acid, commonly called Vitamin A, and in particular retinol, have been used for the treatment of various skin problems, in particular acne, and for combating ageing and dryness of the skin (U.S. Pat. Nos. 4,603,146 and 4,877,805).

The experience acquired in the field of the formulation of products containing retinoids has made it clear that it is difficult to obtain products wherein these active ingredients have a significant shelf life for these active ingredients. Indeed, these agents have a tendency to become decomposed, resulting in their inactivation.

A plurality of formulations, comprising oil-in-water emulsions or using antioxidants, have been proposed to deal with this problem. Such compositions have the advantage of offering adequate quantities of retinoids in active form by virtue of an improvement in the chemical stability of the composition. There may be mentioned, for example, oil-in-water emulsions containing retinoic acid (U.S. Pat. Nos. 3,906,108; 4,247,547; 4,826,828; 4,720,353; EP 143 444 and EP 330 196). This problem of chemical stability of retinoids has led to the development of further improved formulations, in particular to water-in-oil emulsions containing an antioxidant and a chelator (U.S. Pat. No. 5,652,263).

While the solutions proposed in the state of the art have been found satisfactory for cosmetic products such as creams, milks and emulsions, they were found to be inadequate for make-up products containing coloured pigments.

Indeed, it has been found, in the context of the present invention, that retinol is unstable in the presence of metal ions. Combining retinol in a make-up base containing iron and titanium oxides leads to increased instability of retinoids and in particular of retinal, than in the absence of pigments.

Unexpectedly, it has been found that treating the pigments with lipophilic or hydrophilic compounds made it possible to avoid the pigment degrading the retinol: the stability of the retinol is equivalent to that in a base free of pigments.

In addition, protecting one's skin is a daily occupation, requiring a certain amount of time specifically devoted thereto and further requiring the use of several cosmetic products, which may constitute a constrain for consumers. For women, the use of various make-up products may be added to this daily routine. It would therefore be useful to have available make-up products, which would also exert a beneficial effect on the skin and which could therefore replace the specific products for treating the skin mentioned above.

The importance of the invention therefore consists in developing a means for formulating pigmented anti-ageing care products with an active agent whose efficacy is recognized, which makes it possible to satisfy the needs explained above.

### DESCRIPTION OF THE INVENTION

Thus, the present invention relates to a cosmetic composition comprising a retinoid and at least one coloured pigment selected from metal oxides, the said oxides being treated with one or more hydrophilic or lipophilic components.

In another aspect this invention concerns a cosmetic composition comprising a retinoid and at least one coloured pigment selected from metal oxides, the said oxides being coated with one or more hydrophilic or lipophilic components. In embodiments according to this aspect of the invention, the hydrophilic or lipophilic components will be at the surface or concentrated at the surface portion of the pigment particles. The surface of the pigment particles may be coated completely or in part.

As used herein, the term ‘coloured pigment’ is meant to also include white or off-white pigments.

Preferably, the pigments are selected from (red, yellow and black) iron oxides, titanium oxide and dioxide, including mixtures thereof. Particular examples of these pigments are described in Handbook of Pharmaceutical Excipients, The Pharmaceutical Press, Second Ed., pages 131 and 529.

In preferred embodiments the pigments are in the form of particulate material that has been treated or coated as described herein. The pigments more in particular are composed of small particles.

The shape of the pigment particles in said preferred embodiments usually is spherical or essentially spherical although other shapes may also be used, including mixtures of different shapes and sizes. The size of the untreated pigment particles may vary, but in particular sizes up to 100 μm can be used. In particular embodiments, the particle sizes are in the range of about 0.1 to 50 μm, more in particular in the range of about 1 to 50 μm. In other embodiments, pigments with smaller particle sizes can be used, e.g. having particle sizes that are in the range of 5 to 1000 nm, e.g. pigments with very small particle size which is in the range of 5 to 100 nm, or small-sized particles in the range of 100 nm to 500 nm, or pigments with particles sized in the range of 500 to 1000 nm. As used herein ‘particle size’ refers to average particle sizes.

The pigments are treated so that they comprise the hydrophilic or lipophilic component(s). These components may be bound to the pigment either covalently or by other types of binding such as, for example, by electrostatic, Van der Waals or other types of interaction or a combination thereof.

Preferably, the components are bound covalently.

In particular embodiments, the components are hydrophobic. In that instance, the hydrophobically treated pigments may optionally be treated further with suitable oily components.

Examples of components for treating pigments as well as examples of pigments treated with lipophilic agents are described in U.S. Pat. No. 5,897,868. Further examples of components useful to treat pigments may be selected from the components described in U.S. Pat. Nos. 4,606,914, 4,623,074, 4,863,800, JP 60-69011 and JP 61-73775.

The following are examples of hydrophilic or lipophilic components that may be used to treat the pigments for use in the present invention:
Silicones, in particular dimethicone polymers and co-polymers;
Silicon derivatives, in particular trialkoxysilicon groups that are covalently linked to the pigments;
Acrylate or derivatized acrylate polymers and copolymers, for example polyacrylate or polymethacrylate polymers or copolymers;
Fats or oils, in particular triglycerides, more in particular saturated triglycerides; the fats or oils may be derived from natural oils or produced synthetically;
titanate derivatives, in particular acyltitanate and coordinate titanate groups that are covalently linked to the pigments;
fatty acids, in particular C₆₋₃₀-fatty acids, more in particular C₁₂₋₂₄-fatty acids, further in particular C₁₆₋₂₀-fatty acids; specific examples of fatty acids which can be used to this purpose are lauric acid, myristic acid, palmitic acid, oleic acid, stearic acid, isostearic acid, and hydroxystearic acid;
amino acids and derivatives thereof, in particular alkylated and/or acylated amino acid derivatives; of particular interest are N-acyl-amino acids or N-acyl-N-alkyl amino acids wherein the acyl group is derived from a fatty acid, more in particular from a C₁₂₋₂₄-fatty acid, further in particular from a C₁₆₋₂₀-fatty acid, e.g. from capric, lauric, myristic, palmitic, stearic, isostearic, and hydroxystearic, oleic acid; and wherein the alkyl group is lower alkyl, more in particular C₁₋₄-lower alkyl, still more in particular methyl; specific examples comprise N-acyl-L-glutamic acid, N-acyl-N-methylglycine and N-acyl-N-methyl-beta-alanine, wherein the acyl is as mentioned above; of particular interest are lysine and acyl lysine;
proteins or protein derivatives such as collagen, or collagen derivatives, in particular acylated collagen;
phosphatides such as lecithin or derivatives thereof such as hydrogenated lecithin; lecithin can be obtained from natural sources or by synthesis; preferred is hydrogenated lecithin, which may be obtained, for example, from the extraction of lecithin from soybean oil or maize oil, followed by hydrogenation;
peptides, or peptide derivatives, in particular acyl peptides.

The component for treating the pigment is present in the treated pigment in varying amounts. In particular said amount is in the range of about 0.1 to 30%, more in particular from about 0.2 to 20%, still more in particular from about 0.5 to 10%, further in particular from about 1 to 5%, or 1 to 3%, e.g. about 2%, w/w relative to the total weight of the treated pigment.

The treated pigments may be obtained by treating the pigments with suitable quantities of hydrophilic or lipophilic components, said quantities being in the range of about 0.1 to 30%, more in particular from about 0.2 to 20%, still more in particular from about 0.5 to 10%, further in particular from about 1 to 5%, or 1 to 3%, e.g. about 2%, w/w relative to the total weight of the untreated pigment. Or the quantities can be calculated in relation to the quantity of component in the treated pigment which can be as mentioned in the preceding paragraph.

The pigments may be mixed before treatment or treatment may also be carried out on pigments coated beforehand.

Also composite or mixed pigments may be subjected to a treatment. For example, EP 0612516 describes composite or mixed pigments of titanium dioxide and iron oxides as well as methods for the manufacture thereof.

A TiO₂-iron oxide composite oxide treated with an alkylsilane (trialkoxyoctylsilane) is available from Degussa under the reference T 817. This pigment is of particular interest since its size confers advantageous anti-UV properties on it making it useful for sunscreen products.

The pigments may be treated according to the methodologies described in the references cited herein.

One particular way of treating the pigments with acidic components is by using appropriate metal or ammonium salts. Treatment with fatty acids, amino acid derivatives, acrylic acid polymers or copolymers, and the like, may be done by treatment of the pigments with the alkali metal, alkaline earth metal, aluminum or transition metal salts of these acids or acid derivatives. Particularly interesting salts are the Na, K, Mg, Ca, Al, Zn, Zr or Ti salts.

In a preferred embodiment, the compositions contain titanate coated pigments. The latter are pigments that have been treated with suitable titanate coupling agents.

Suitable titanate coupling agents comprise monoalkoxy titanates and coordinate titanates.

The former in particular are monoalkoxy triacyl titanates. These are also referred to as monoalkyl titanates and may be represented by the formula RO—Ti—(OR′)₃, wherein R is alkyl and R′ is a suitable organic radical which can be the same or different.

In certain embodiments of these titanates, alkoxy is C₁₋₆-alkyloxy, in particular is C₁₋₄-alkyloxy and acyl is derived from acrylic acid or from an acrylic acid derivative, e.g. methacrylic acid, or from a fatty acid. Acyl groups in particular are derived from a C₆₋₃₀-fatty, more in particular from a C₁₂₋₂₄-fatty acid, further in particular from a C₁₆₋₂₀-fatty acid, e.g. from capric, lauric, myristic, palmitic, stearic, isostearic, hydroxy stearic, oleic acid. The acyl groups in these triacyl titanates can be the same or different. Preferred embodiments are mono-isopropyl triacyl titanates.

Suitable coordinate titanates comprise for example phosphito titanate complexes. These comprise the complexes described and mentioned in U.S. Pat. No. 4,261,913 which are adducts of a disubstituted hydrogen phosphite with a tetrasubstituted titanate.

Of particular interest are the monoalkoxy triacyl titanates and coordinate titanates described and mentioned in U.S. Pat. No. 4,877,604.

As particularly suitable monoalkoxy titanates, there may be mentioned for example isopropyl triisostearoyl titanate (‘ITT’, available from KOBO), isopropyl dimethacryl isostearoyl titanate, isopropyl dimethacryl isostearoyl titanate. As particularly suitable coordinated titanates, there may be mentioned tetraisopropyl di(dioctyl)phosphito titanate and tetra(2,2-diallyoxymethyl)butyl, di(ditridecyl)phosphito titanate.

The treatment with these titanates is lipophilic and the thus treated pigments can easily be dispersed in oils.

The quantity of titanate used may vary and can for example be from 0.01 to 5.0%, in particular from 0.1 to 2% by weight of the treated pigment. The titanate treated pigment equally contains from about 0.01 to about 5.0%, in particular from about 0.1 to about 2%, all w/w (relative to the total weight of the treated pigment) of titanate component.

Titanate coated pigments can be obtained by art-known methodologies and in particular by the methodologies described in U.S. Pat. No. 4,877,604. In the latter reference two types of procedures are mentioned that resulted in the same quality of coating on (yellow, black and red) iron oxides and on titanium dioxide.

These two procedures are:
a) the addition of a titanate, which in particular is a monoalkoxy titanate, in liquid form, to a dispersion containing about 15 to 20% of pigments, with rapid stirring at room temperature, followed by filtration; the coated pigments are then recovered (washing and drying of the filter paper);
b) spraying of a titanate, which in particular is a monoalkoxy titanate, in liquid form, onto a fluidised or agitated filler bed of pigments, followed by mixing.

The cosmetic composition defined above may therefore comprise at least one coloured pigment selected from monoalkoxylated metal oxides, in particular monoalkoxy titanates and metal oxides (for example titanates) coordinated with four C6 to C18 carbon chains comprising heteroatoms, in particular a phosphite or phosphate.

Among the coated pigments available from KOBO, the following titanates are more particularly used:

| | | | % |
|---|---|---|---|
| Reference | INCI name | Coating | coating |
| BBO-12 | Iron oxide (98%) & Isopropyl | Monoalkoxy | 2 |
| | Titanium Triisostearate | titanate | |
| BYO-12 | Iron oxide (98%) & ITT (2%) | Id | 2 |
| BRO-12 | Iron oxide (98%) & ITT (2%) | Id | 2 |
| BTO-12 | Titanium dioxide (98%) & ITT | Id | 2 |
| | (2%) | | |
| BBO-IIS2 | Iron oxide & Triethoxy | Silicone | 2 |
| | caprylsilane | (Silane) | |
| BBO/ | Iron oxide & Magnesium myristate | Salt of a | 1.5 |
| MM1.5 | | polyvalent | |
| | | metal and a | |
| | | fatty acid | |
| BBO/ | Iron oxide & Magnesium myristate | Id | 3 |
| MM3 | | | |
| WE55Y | BYO-I2 & polyglyceryl-4 | BYO-12 (45%) | |
| | isostearate & cetyl dimethicone & | | |
| | hexyl laurate | | |
| WE70B | BBO-I2 & polyglyceryl-4 | BBO-12 (70%) | |
| | isostearate & cetyl dimethicone & | | |
| | hexyl laurate | | |

In an additional particular embodiment, the invention relates to a cosmetic composition mentioned above in which the metal oxides treated or coated with one or more hydrophilic or lipophilic compounds have anti-UV properties.

Among the retinoids for use in the compositons of this invention, retinal is of particular interest. Nevertheless, the invention may also be carried out with retinoic acid, retinal, retinyl esters and retinyl palmitate.

The compositions according to the invention are characterized by its properties of good chemical and physical stability. Indeed, after 15 days at 50° C., the composition still comprises at least 60%, 70% or preferably 80% retinol.

This good stability is due to treating of the pigments. Particularly attractive coatings are based on isopropyl titanium triisostearate (ITT), ammonium polyacrylate, lauroyl lysine or a silica+dimethicone+methicone mixture.

In another aspect, the composition defined above exists in the form of an oil-in-water emulsion, a water-in-oil emulsion or a multiple emulsion.

It may also exist in anhydrous form, in particular in the case where the final product is a foundation. It may also be provided in a form wherein the active is formulated in liposomes or any other vehicle suitable for cosmetic application.

Advantageously, the composition comprises from 0.001% to 1%, preferably from 0.01% to 0.15% of retinol by weight relative to the finished product and from 0.5% to 30%, preferably from 2% to 15% of pigments by weight relative to the finished product.

It may comprise, in addition, stabilizers selected from sequestering agents and antioxidants, which may be added to further increase the shelf life of the retinoid.

Moreover, the composition may comprise sunscreen agents, vitamins, in particular Vitamin C, and oils, in particular oils with an iodine value greater than 70 or any other ingredient which is useful in cosmetics, in particular silicones.

In another aspect, the invention relates to the use of a composition mentioned above as a two-in-one product for use as a makeup and for the treatment of ageing of the skin and of the aging spots.

Preferably, the composition may be used in a variety of cosmetic formulations such as cakes, foundations, mascaras, eye shadows, eye liners, pens, blushes, powders, moisturizers, tinted creams, lotions, compact forms, lipsticks. These make-up products may also serve as sunscreen agents.

All references cited herein are incorporated herein in their entirety.

The following examples are aimed at illustrating the invention, not to limit it thereto.

### EXAMPLE 1

### Stability of Retinol in the Presence of Coloured Pigments

1.1 Cosmetic base A containing retinol (concentration 0.044%)

In this and the following examples % are w/w relative to the total weight of the formulation.

| | % retinol at 15 | |
|---|---|---|
| Composition | days at 50° C. | CONCLUSIONS |
| Base A without pigment | 65% +/− 3% | |
| Base A with Kobo coated | 61% +/− 3% | Coated pigments: |
| pigments | | protection of the |
| | | retinol |
| Base A with uncoated | 53% +/− 0% | Uncoated pigments: |
| pigments | | the retinol is |
| | | destabilized by the |
| | | presence of the |
| | | pigments |

1.2 Cosmetic base B containing retinol (concentration: 0.044%)

| | % retinol at 15 | |
|---|---|---|
| Composition | days at 50° C. | CONCLUSIONS |
| Base B without pigment | 82% +/− 2% | |
| Base B with Kobo coated | 78% +/− 2% | No significant |
| pigments | | difference compared |
| | | with a placebo: the |
| | | coating protects the |
| | | retinol |

On 2 different bases, it is confirmed that the coating of the pigments makes it possible to have a similar result for the stability of the retinol.
Base B is better than base A because additives were added which make it possible to have a better retinol stability. Base B contains EDTA (hydrophilic sequestrant) and BHT (lipophilic antioxidant).

The two series of experiments were carried out with the same quantity of coating on (yellow, black and red) iron oxides and on titanium oxide. The treatment is hydrophilic and is with isopropyl titanium triisostearate (ITT). The supplier of these pigments is KOBO.

1. Cosmetic composition comprising a retinoid and at least one coloured pigment selected from metal oxides, the said oxides being treated with one or more hydrophilic or lipophilic components.

2. Composition according to claim 1, wherein the metal oxides are are selected from (red, yellow and black) iron oxides, titanium dioxide, including mixtures thereof.

3. Composition according to claim 1 or 2, characterized in that the metal oxides are treated with one or more silicones; silicon derivatives, acrylate or derivatized acrylate polymers and copolymers, fats or oils, titanate derivatives, fatty acids, amino acids and derivatives thereof, proteins or protein derivatives, phosphatides, peptides, or peptide derivatives.

4. Composition according to claim 3, characterized in that the metal oxides are treated with one or more silicones; silicon derivatives, acrylate or derivatized acrylate polymers and copolymers, fats or oils, titanate derivatives, fatty acids, acyl amino acids, proteins or protein derivatives, hydrogenated lecithin, acyl peptides.

5. Composition according to claim 4, characterized in that the metal oxides are treated with one or more fatty acids, acylamino acids, hydrogenated lecithin, acyl collagen, or titanate derivatives.

6. Composition according to claim 5, characterized in that the metal oxides are treated with monoalkyl titanates or coordinate titanates.

7. Composition according to claim 5, characterized in that the metal oxides are treated with monoalkyl triacyl titanates.

8. Composition according to claim 5, characterized in that the metal oxides are treated with isopropyl titanium triisostearate (ITT).

9. Composition according to any of claims 1 to 8, wherein the component for treating the pigment is present in an amount which is in the range of about 0.1 to 30%, in particular from about 0.2 to 20%, more in particular from about 0.5 to 10%, further in particular from about 1 to 5%, w/w relative to the total weight of the treated pigment.

10. Composition according to any of claims 1 to 9, characterized in that the retinoid is retinol.

11. Composition according to any of claims 1 to 10, characterized in that it is provided in the form of an oil-in-water emulsion, a water-in-oil emulsion or multiple emulsions.

12. Composition according to to any of claims 1 to 10, characterized in that it is provided in anhydrous form.

13. Composition according any of the preceding claims, characterized in that it comprises from 0.001% to 1%, preferably from 0.01% to 0.15% of retinol by weight relative to the finished product.

14. Composition according to any of the preceding claims, characterized in that it comprises from 0.5% to 30%, preferably from 2% to 15% of pigments by weight relative to the finished product.

15. Composition according to any of the preceding claims, characterized in that it comprises oils, in particular oils with an iodine value greater than 70.

16. Use of a composition according to any of the preceding claims, as a two-in-one product for use as a make-up product that has also activity against the effects of skin-ageing and aging spots.

17. Use according to claim 16, as foundation, tinted creams, compact forms and lipstick.

18. Use according to claim 17, as a make-up product with sunscreen properties.
